SEQUENCE LISTING (27 sequences)

SEQ ID NO: 1 - DNA, 1734 bp, Homo sapiens
  CDS (71)..(1618): Human Nox1 polypeptide of SEQ NO2
ggacctctccagaatccggattgctgaatcttccctgttgcctagaagggctccaaaccacctcttgacaatgggaaactgggtggttaaccactggttttcagttttgtttctggttgtttggttagggctgaatgttttcctgtttgtggatgccttcctgaaatatgagaaggccgacaaatactactacacaagaaaaatccttgggtcaacattggcctgtgcccgagcgtctgctctctgcttgaattttaacagcacgctgatcctgcttcctgtgtgtcgcaatctgctgtccttcctgaggggcacctgctcattttgcagccgcacactgagaaagcaattggatcacaacctcaccttccacaagctggtggcctatatgatctgcctacatacagctattcacatcattgcacacctgtttaactttgactgctatagcagaagccgacaggccacagatggctcccttgcctccattctctccagcctatctcatgatgagaaaaaggggggttcttggctaaatcccatccagtcccgaaacacgacagtggagtatgtgacattcaccagcattgctggtctcactggagtgatcatgacaatagccttgattctcatggtaacttcagctactgagttcatccggaggagttattttgaagtcttctggtatactcaccacctttttatcttctatatccttggcttagggattcacggcattggtggaattgtccggggtcaaacagaggagagcatgaatgagagtcatcctcgcaagtgtgcagagtcttttgagatgtgggatgatcgtgactcccactgtaggcgccctaagtttgaagggcatccccctgagtcttggaagtggatccttgcaccggtcattctttatatctgtgaaaggatcctccggttttaccgctcccagcagaaggttgtgattaccaaggttgttatgcacccatccaaagttttggaattgcagatgaacaagcgtggcttcagcatggaagtggggcagtatatctttgttaattgcccctcaatctctctcctggaatggcatccttttactttgacctctgctccagaggaagatttcttctccattcatatccgagcagcaggggactggacagaaaatctcataagggctttcgaacaacaatattcaccaattcccaggattgaagtggatggtccctttggcacagccagtgaggatgttttccagtatgaagtggctgtgctggttggagcaggaattggggtcaccccctttgcttctatcttgaaatccatctggtacaaattccagtgtgcagaccacaacctcaaaacaaaaaaggttggtcatgcagcattaaactttgacaaggccactgacatcgtgacaggtctgaaacagaaaacctcctttgggagaccaatgtgggacaatgagttttctacaatagctacctcccaccccaagtctgtagtgggagttttcttatgtggccctcggactttggcaaagagcctgcgcaaatgctgtcaccgatattccagtctggatcctagaaaggttcaattctacttcaacaaagaaaatttttgagttataggaataaggacggtaatctgcattttgtctctttgtatcttcagtaatttacttggtctcgtcaggtttgagcagtcactttaggataagaatgtgcctctcaagccttg

SEQ ID NO: 2 - protein, 515 aa, Homo sapiens
MGNWVVNHWFSVLFLVVWLGLNVFLFVDAFLKYEKADKYYYTRKILGSTLACARASALCLNFNSTLILLPVCRNLLSFLRGTCSFCSRTLRKQLDHNLTFHKLVAYMICLHTAIHIIAHLFNFDCYSRSRQATDGSLASILSSLSHDEKKGGSWLNPIQSRNTTVEYVTFTSIAGLTGVIMTIALILMVTSATEFIRRSYFEVFWYTHHLFIFYILGLGIHGIGGIVRGQTEESMNESHPRKCAESFEMWDDRDSHCRRPKFEGHPPESWKWILAPVILYICERILRFYRSQQKVVITKVVMHPSKVLELQMNKRGFSMEVGQYIFVNCPSISLLEWHPFTLTSAPEEDFFSIHIRAAGDWTENLIRAFEQQYSPIPRIEVDGPFGTASEDVFQYEVAVLVGAGIGVTPFASILKSIWYKFQCADHNLKTKKVGHAALNFDKATDIVTGLKQKTSFGRPMWDNEFSTIATSHPKSVVGVFLCGPRTLAKSLRKCCHRYSSLDPRKVQFYFNKENF

SEQ ID NO: 3 - DNA, 2577 bp, Rattus norvegicus
  CDS (128)..(1819): Rat Nox1 polypeptide of SEQ NO4
ttctgagtaggtgtgcatttgagtgtcataaagacatatatcttgagctagacagaagttcctatcctgaaggatcccatcagagaaaccagattgctcctaagaggctccagacctccatttgacaatgggaaactggctggttaaccactggctctcagttttgtttctggtttcttggttggggctgaacatttttctgtttgtgtacgtcttcctgaattatgagaagtctgacaagtactattacacgagagaaattctcggaactgccttggccttggccagagcatctgctttgtgcctgaattttaacagcatggtgatcctgattcctgtgtgtcgaaatctgctctccttcctgaggggcacctgctcattttgcaaccacacgctgagaaagccattggatcacaacctcaccttccataagctggtggcatatatgatctgcatattcacagctattcatatcattgcacatctatttaactttgaacgctacagtagaagccaacaggccatggatggatctcttgcctctgttctctccagcctattccatcccgagaaagaagattcttggctaaatcccatccagtctccaaacgtgacagtgatgtatgcagcatttaccagtattgctggccttactggagtggtcgccactgtggctttggttctcatggtaacttcagctatggagtttatccgcaggaattattttgagctcttctggtatacacatcaccttttcatcatctatatcatctgcttagggatccatggcctgggggggattgtccggggtcaaacagaagagagcatgagtgaaagtcatccccgcaactgttcatactctttccacgagtgggataagtatgaaaggagttgcaggagtcctcattttgtggggcaaccccctgagtcttggaagtggatcctcgcgccgattgctttttatatctttgaaaggatccttcgcttttatcgctcccggcagaaggtcgtgattaccaaggttgtcatgcacccatgtaaagttttggaattgcagatgaggaagcggggctttactatgggaataggacagtatatattcgtaaattgcccctcgatttccttcctggaatggcatccctttactctgacctctgctccagaggaagaatttttctccattcatattcgagcagcaggggactggacagaaaatctcataaggacatttgaacaacagcactcaccaatgcccaggatcgaggtggatggtccctttggcacagtcagtgaggatgtcttccagtacgaagtggctgtactggttggggcagggattggcgtcactccctttgcttccttcttgaaatctatctggtacaaattccagcgtgcacacaacaagctgaaaacacaaaagatctatttctactggatttgtagagagacgggtgcctttgcctggttcaacaacttattgaattccctggaacaagagatggacgaattaggcaaaccggatttcctaaactaccgactcttcctcactggctgggatagcaacattgctggtcatgcagcattaaactttgacagagccactgacgtcctgacaggtctgaaacagaaaacctcctttgggagaccaatgtgggacaatgagttttctagaatagctactgcccaccccaagtctgtggtgggggttttcttatgcggccctccgactttggcaaaaagcctgcgcaaatgctgtcggcggtactcaagtctggatcctaggaaggttcaattctacttcaacaaagaaacgttctgaattggaggaagccgcacagtagtacttctccatcttccttttcactaacgtgtgggtcagctactagatagtccgttgtcgcacaaggacttcactcccatcttaaagttgactcaactccatcattcttgggctttggcaacatgagagctgcataactcacaattgcaaaacacatgaattattattggggggattgtaaatccttctgggaaacctgcctttagctgaatcttgctggttgacacttgcacaatttaacctcaggtgtcttggttgatacctgataatcttccctcccacctgtccctcacagaagatttctaagtagggtgattttaaaatatttattgaatccacgacaaaacaataatcataaataataaacataaaattaccaagattcccactcccatatcatacccactaagaacatcgttatacatgagcttatcatccagtgtgaccaacaatttatactttactgtgccaaaataatcttcatctttgcttattgaacaattttgctgactttccctagtaatatcttaagtatattaactggaatcaaatttgtattatagttagaagccaactatattgccagtttgtattgtttgaaataactggaaaggcctgacctacatcgtggggtaatttaacagaagctctttccattttttgttgttgttgttaaagagttttgtttatgaatgtgttataaaaagaaaataaaaagttataattttgacggaaaa

SEQ ID NO: 4 - protein, 563 aa, Rattus norvegicus
MGNWLVNHWLSVLFLVSWLGLNIFLFVYVFLNYEKSDKYYYTREILGTALALARASALCLNFNSMVILIPVCRNLLSFLRGTCSFCNHTLRKPLDHNLTFHKLVAYMICIFTAIHIIAHLFNFERYSRSQQAMDGSLASVLSSLFHPEKEDSWLNPIQSPNVTVMYAAFTSIAGLTGVVATVALVLMVTSAMEFIRRNYFELFWYTHHLFIIYIICLGIHGLGGIVRGQTEESMSESHPRNCSYSFHEWDKYERSCRSPHFVGQPPESWKWILAPIAFYIFERILRFYRSRQKVVITKVVMHPCKVLELQMRKRGFTMGIGQYIFVNCPSISFLEWHPFTLTSAPEEEFFSIHIRAAGDWTENLIRTFEQQHSPMPRIEVDGPFGTVSEDVFQYEVAVLVGAGIGVTPFASFLKSIWYKFQRAHNKLKTQKIYFYWICRETGAFAWFNNLLNSLEQEMDELGKPDFLNYRLFLTGWDSNIAGHAALNFDRATDVLTGLKQKTSFGRPMWDNEFSRIATAHPKSVVGVFLCGPPTLAKSLRKCCRRYSSLDPRKVQFYFNKETF

SEQ ID NO: 5 - DNA, 20 bp, Artificial sequence
  Forward primer for Nox1 gene
ggagcaggaattggggtcac

SEQ ID NO: 6 - DNA, 20 bp, Artificial sequence
  Reverse primer for Nox1 gene
ttgctgtcccatccggtgag

SEQ ID NO: 7 - DNA, 21 bp, Artificial sequence
  Forward primer for human Nox1 gene
ccactgtaggcgccctaagtt

SEQ ID NO: 8 - DNA, 20 bp, Artificial sequence
  Reverse primer for human Nox1 gene
aagaatgaccggtgcaagga

SEQ ID NO: 9 - DNA, 25 bp, Artificial sequence
  TaqMan probe
aagggcatccccctgagtcttggaa

SEQ ID NO: 10 - DNA, 58 bp, Artificial sequence
  siRNA for human Nox1 gene
gcgtggcttcagcatggaattcaagagattccatgctgaagccacgcttttttggaaa

SEQ ID NO: 11 - DNA, 58 bp, Artificial sequence
  siRNA for human Nox1 gene
gggctttcgaacaacaatattcaagagatattgttgttcgaaagcccttttttggaaa

SEQ ID NO: 12 - DNA, 59 bp, Artificial sequence
  siRNA for rat Nox1 gene
gttatgagaagtctgacaagttcaagagacttgtcagacttctcataattttttggaaa

SEQ ID NO: 13 - DNA, 58 bp, Artificial sequence
  siRNA for rat Nox1 gene
gattcttggctaaatcccattcaagagatgggatttagccaagaatcttttttggaaa

SEQ ID NO: 14 - DNA, 58 bp, Artificial sequence
  siRNA for rat Nox1 gene
ggacatttgaacaacagcattcaagagatgctgttgttcaaatgtccttttttggaaa

SEQ ID NO: 15 - DNA, 20 bp, Artificial sequence
  Forward primer for Nox1 gene
ggtcactccctttgcttcca

SEQ ID NO: 16 - DNA, 21 bp, Artificial sequence
  Reverse primer for rat Nox1 gene
ggcaaaggcacctgtctctct

SEQ ID NO: 17 - DNA, 20 bp, Artificial sequence
  TaqManMGB probe
tccagtagaaatagatcttt

SEQ ID NO: 18 - DNA, 65 bp, Artificial sequence
  siRNA construction
gatcccgttatgagaagtctgacaagttcaagagacttgtcagacttctcataattttttggaaa

SEQ ID NO: 19 - DNA, 65 bp, Artificial sequence
  siRNA construction
agcttttccaaaaaattatgagaagtctgacaagtctcttgaacttgtcagacttctcataacgg

SEQ ID NO: 20 - DNA, 64 bp, Artificial sequence
  siRNA construction
gatcccgattcttggctaaatcccattcaagagatgggatttagccaagaatcttttttggaaa

SEQ ID NO: 21 - DNA, 64 bp, Artificial sequence
  siRNA construction
agcttttccaaaaaagattcttggctaaatcccatctcttgaatgggatttagccaagaatcgg

SEQ ID NO: 22 - DNA, 64 bp, Artificial sequence
  siRNA construction
gatcccggacatttgaacaacagcattcaagagatgctgttgttcaaatgtccttttttggaaa

SEQ ID NO: 23 - DNA, 64 bp, Artificial sequence
  siRNA construction
agcttttccaaaaaaggacatttgaacaacagcatctcttgaatgctgttgttcaaatgtccgg

SEQ ID NO: 24 - DNA, 17 bp, Artificial sequence
  M13 primer
gttttcccagtcacgac

SEQ ID NO: 25 - DNA, 21 bp, Artificial sequence
  3.0 Rev primer
gagttagctcactcattaggc

SEQ ID NO: 26 - DNA, 19 bp, Artificial sequence
  Forward primer for Nox1 gene
atgggaaactggctggtta

SEQ ID NO: 27 - DNA, 21 bp, Artificial sequence
  Reverse primer for Nox1 gene
tcagaacgtttctttgttgaa

(19) United States
(12) Patent Application Publication
(10) Pub. No.: US 2007/0009509 A1
(43) Pub. Date: Jan. 11, 2007

(54) Antibody against nox1 polypeptide, method of diagnosing cancer with the use of nox1 gene and method of screening cancer growth inhibitor

(76) Inventors: Junji Mitsushita, Nagano (JP); Toru Kamata, Nagano (JP); Kunitaka Hirose, Tokyo (JP)

Correspondence Address: REED SMITH LLP, 3110 FAIRVIEW PARK DRIVE, FALLS CHURCH, VA, 22042, US

(21) Appl. No.: 10/570,011

(22) PCT Filed: Aug. 6, 2004
(86) PCT No.: PCT/JP04/11673

§ 371 Date: Jul. 19, 2006

(30) Foreign Application Priority Data
Sep. 1, 2003 (JP) 2003-308658

(51) Int. Cl. A61K 39/395; C12Q 1/68; G01N 33/574

(52) U.S. Cl. 424/133.1; 424/155.1; 435/6; 435/7.23

(57) ABSTRACT

The present invention provides a diagnostic method for cancer, a screening method for a cancer growth inhibitor, and a pharmaceutical composition used in cancer therapy using a Nox1 gene associated with a mutant Ras oncogene. More specifically, the present invention relates to: a composition for producing an antibody, comprising a polypeptide coded for a Nox1 gene, a homologue thereof, and their peptide fragments; an antibody against the polypeptide coded for a Nox1 gene; and a method for detecting the antibody or Nox1-expressing mRNA.

### TECHNICAL FIELD

The present invention relates to a diagnostic method for cancer, a screening method for a cancer growth inhibitor, and a pharmaceutical composition based on a cancer-associated gene and a polypeptide coded for the gene. More specifically, the present invention relates to a diagnostic method for cancer; a screening method for a cancer growth inhibitor; and a pharmaceutical composition used in cancer therapy, using a Nox1 gene associated with a mutant Ras oncogene, a polypeptide coded for the gene, and an antibody specific to the polypeptide.

### BACKGROUND ART

A Mutant Ras oncogene is heretofore known to exert great influence on mammalian cell carcinogenesis and its progression (Non-Patent Document 1). For example, the mutant Ras oncogene is considered to cancerate animal cells and aggravate the cancer via a Raf-MAPKK-MAPK pathway, one of its principal downstream pathways (Non-Patent Document 2).

Based on these findings, there have been developed a method for tumor suppressor gene therapy comprising administering an expression vector gene comprising a gene coding for p94RB to a targeted cancer cell depleted of N-ras tumor suppressor (Patent Document 1), and a method of identifying a tumor suppressor gene by use of H-ras, K-ras, and N-ras oncogenes (Patent Document 2).

Recent research has reported that cells transformed with mutant Ras gene produce reactive oxygen species (ROS) such as superoxide and H₂O₂ (Non-Patent Document 3). Low-level intracellular ROS plays a role as a signal molecule in growth factor-induced cell growth (Non-Patent Documents 4 and 5), suggesting that a rise in ROS generation associated with mutant Ras gene possibly causes the abnormal growth of animal cells.

However, reactive oxygen-producing enzymes that are involved in carcinogenesis, cancer promotion, and so on, caused by mutant Ras gene have heretofore been unknown.
(Patent Document 1) National Publication of International Patent Application No. 1996-508166
(Patent Document 2) National Publication of International Patent Application No. 1998-504448
(Non-Patent Document 1) Lowy, D. R. Annu. Rev. Biochem. 62, 851-891 (1993)
(Non-Patent Document 2) McCormick, F. TCB. 9, M53-M56 (1999)
(Non-Patent Document 3) Irani, K. et al. Science 275, 1649-1652 (1997)
(Non-Patent Document 4) Sundaresan, M. et al. Science. 270, 296-299 (1995)
(Non-Patent Document 5) Bae, Y. S. et al. J. Biol. Chem. 272, 217-221 (1997)

### DISCLOSURE OF THE INVENTION

An object of the present invention is to provide a diagnostic method for cancer and a screening method for a cancer growth inhibitor; and a pharmaceutical composition used in cancer therapy using a Nox-1 gene associated with a mutant Ras oncogene, a polypeptide coded by the gene, and an antibody specific to the polypeptide.

The present inventors conducted studies and consequently gained findings that a mutant Ras oncogene remarkably increases the expression of Nox1 (1, 2, and 3), a homologue of the catalytic subunit of superoxide-generating NADPH oxidase, in an MAPKK-MAPK pathway, and that siRNA of the Nox1 gene effectively suppresses phenotypes of a mutant Ras gene such as cell adhesion-dependent growth, morphological changes, and tumor formulation in athymic mice. Based on the findings, the present inventors attained the object and completed the present invention.

Namely, the present invention provides a composition for producing an antibody, comprising: (1) a polypeptide comprising the amino acid sequence of SEQ ID NO: 2; (2) a polypeptide having an amino acid sequence mutated from the amino acid sequence of SEQ ID NO: 2 by the substitution, deletion, addition, and/or insertion of one or more amino acid residues and inducing the production of an antibody specific to the polypeptide comprising the amino acid sequence of SEQ ID NO: 2; or (3) a polypeptide fragment having a partial sequence of the polypeptide of (1) or (2) and inducing the production of an antibody specific to the polypeptide comprising the amino acid sequence of SEQ ID NO: 2.

Moreover, the present invention provides a method for producing an antibody specific to a polypeptide comprising the amino acid sequence of SEQ ID NO: 2, comprising administering the composition for producing an antibody to a mammal.

Furthermore, the present invention provides an antibody specific to a polypeptide comprising the amino acid sequence of SEQ ID NO: 2.

Furthermore, the present invention provides a diagnostic method for cancer, comprising bringing the antibody specific to a polypeptide comprising the amino acid sequence of SEQ ID NO: 2 into contact with a biological sample.

Furthermore, the present invention provides a diagnostic composition for cancer and a pharmaceutical composition for cancer therapy, comprising the antibody.

Furthermore, the present invention provides a diagnostic method for cancer, characterized by detecting a polynucleotide comprising the nucleotide sequence of SEQ ID NO: 1 or a fragment thereof. It is preferred that the detection should be performed by detecting the polynucleotide or the fragment thereof by polymerase chain reaction or real-time quantitative polymerase chain reaction.

Furthermore, the present invention provides siRNA corresponding to a polynucleotide comprising the nucleotide sequence of SEQ ID NO: 1 or a fragment thereof.

Furthermore, the present invention provides a pharmaceutical composition for cancer therapy, comprising: siRNA corresponding to a polynucleotide comprising the nucleotide sequence of SEQ ID NO: 1 or a fragment thereof; and siRNA corresponding to a polynucleotide comprising a nucleotide sequence at positions from 71 to 1615 of SEQ ID NO: 1 or a fragment thereof.

Furthermore, the present invention provides a pharmaceutical composition for cancer therapy, comprising a cell transformed with siRNA corresponding to a polynucleotide comprising the nucleotide sequence of SEQ ID NO: 1 or a fragment thereof; and siRNA corresponding to a polynucleotide comprising a nucleotide sequence at positions from 71 to 1615 of SEQ ID NO: 1 or a fragment thereof.

Furthermore, the present invention provides a screening method for a cancer cell growth inhibitor targeted to a Nox1 gene, comprising: (1) transfecting a cell with a Nox1 gene; bringing the transformed cell into contact with a substance to be screened; and detecting the inactivation of Nox1 activity (primary screening); and (2) investigating whether or not the Nox1-inactivating substance suppresses cancer cell growth (secondary screening), thereby detecting the expression of the Nox1 gene and the inactivation of Nox1 activity.

### DEFINITION

The term “polypeptide” used herein means a primary structure of an amino acid sequence, a polypeptide fragment with the primary structure, a polypeptide having biological activity accompanied with a three-dimensional structure, or a protein.

A “Nox1 polypeptide” used herein refers to a polypeptide coded for a Nox1 gene and includes both full-length polypeptide and polypeptide fragment.

The term “homologue” used herein refers to a polypeptide having homology in an amino acid sequence to a polypeptide or protein with a particular amino acid sequence and having biological activity or antigenicity in common with the polypeptide or protein.

The term “siRNA” used herein means a short RNA fragment that suppresses the expression of a particular gene or a double-stranded RNA molecule that consists of the RNA fragment and its complementary strand.

A “mutant Ras gene” used herein refers to a human Ras gene in a mutated state that is involved in tumor formation. The mutant Ras gene is, for example, a human Ras gene having a K-Ras mutation of glycine at the 12th codon to aspartic acid or to valine or arginines, which is commonly seen in human pancreatic cancer, as well as a human Ras gene having K-Ras mutations occurring at the 12th codon and additionally at the 13th and 61st codons, which are found in lung cancer.

A “partial sequence of an amino acid sequence” of the polypeptide used herein refers to a sequence that comprises at least 8 or more consecutive amino acid residues contained in the amino acid sequence.

An “oligonucleotide” used herein refers to a nucleotide that typically comprises less than 100 bases and preferably comprises 6 to 99 bases.

### BRIEF DESCRIPTION OF THE DRAWINGS

FIG. 1(a) is a graph showing a result of gene expression analysis conducted by real-time quantitative polymerase chain reaction in Example 1;

FIGS. 1(b), (c), and (d) are, results of detecting Nox1 expression in Example 2 by conducting real-time quantitative polymerase chain reaction in the same way as in Example 1;

FIG. 2 is an electrophoretic image obtained in Example 4 by conducting PCR by use of M13F and 3.0Rev as primers to confirm a result of transformation of cell lines stably transfected with RNAi(1), RNAi(3), or RNAi(5);

FIG. 3 shows photographs obtained in Example 4, wherein morphological changes in which Nox1 gene is involved are compared among cells;

FIG. 4 is a graph showing a result of measuring the adhesion-dependent cell growth of cell lines exhibited by soft agar culture in Example 4 in order to examine the transformation of cells by observing anchorage-independent growth capacity;

FIG. 5 is a graph showing a growth curve obtained in Example 4 by measuring the number of each of K-Ras-NRK/neg-1 (neg-1), K-Ras-NRK/RNAi(1)-7 (i(1)-7), K-Ras-NRK/RNAi(3)-19 (i(3)-19), and K-Ras-NRK/RNAi(5)-7 (i(5)-7) cells in a liquid medium;

FIG. 6 is a graph showing a result of quantifying a decrease in NBT in Example 5 by solubilizing treated cells and measuring absorbance at 510 nm;

FIG. 7 is a graph showing in Example 6 that a GFP-rat Nox1 fusion polypeptide is produced, that the coexpression of RNAi(1), RNAi (3), and RNAi(5) inhibits the production of a GFP-rat Nox1 fusion protein dose-dependently on their vectors (Left panel), and that the RNAi constructs have high specificity to their target gene (right panel);

FIG. 8 is an electrophoretic image obtained in Example 6 by conducting RT-PCR to confirm that the mRNA expression of endogenous Nox1 is inhibited in each of cells, as compared to K-Ras-NRK and K-Ras-NRK/neg-1 cells;

FIG. 9 is an image obtained in Example 7 by immunoblotting by the same approach as in Example 6, wherein the image demonstrates that siRNA used does not inhibit GFP or GFP-human Nox1 expression;

FIG. 10 is a photograph showing the influence of reactivation of Nox1 once inhibited by siRNA on cells in terms of morphological changes in K-Ras-NRK cells in Example 7;

FIG. 11 is a graph showing the influence of reactivation of Nox1 once inhibited by siRNA on cells in terms of cell growth rates;

FIG. 12 is a graph showing a result of investigating adhesion-dependent cell growth capacity by use of a soft agar culture method in order to examine the influence of reactivation of Nox1 once inhibited by siRNA on cells by observing anchorage-independent growth capacity;

FIG. 13 is a graph showing a result of observing morphological changes in cells in order to investigate the influence of Nox1 gene expression on transformation with mutant Ras in Example 9; and

FIG. 14 is a graph showing the influence of siRNA on tumor formation caused by mutant Ras by observing tumor formation in a nude mouse over 1 month and measuring the volume of the formed tumor in Example 9.

### MOST PREFERABLE MODE FOR CARRYING OUT THE INVENTION

A composition for producing an antibody of the present invention comprises: (1) a polypeptide comprising the amino acid sequence of SEQ ID NO: 2; (2) a polypeptide having an amino acid sequence mutated from the amino acid sequence of SEQ ID NO: 2 by the substitution, deletion, addition, and/or insertion of one or more amino acid residues and inducing the production of an antibody specific to the polypeptide comprising the amino acid sequence of SEQ ID NO: 2; or (3) a polypeptide fragment having a partial sequence of the polypeptide of (1) or (2) and inducing the production of an antibody specific to the polypeptide comprising the amino acid sequence of SEQ ID NO: 2.

The composition for producing an antibody of the present invention may contain a polypeptide having at least 80% homology, preferably 90% homology, particularly preferably 95% homology, to the amino acid sequence of SEQ ID NO: 2. The homology of amino acid sequences used herein refers to identity (%) between a reference amino acid sequence and an amino acid sequence compared therewith when the sequences are aligned. The homology can be calculated using default (initial setting) parameters in analytical software such as BLAST (J. Mol. Biol., 215, 403 (1990)) and FASTA (Methods in Enzymology, 183, 63-69) that are standard programs for performing the homology search of nucleotide sequences and amino acid sequences.

The polypeptide comprising the amino acid sequence of SEQ ID NO: 2 is a polypeptide coded for a Nox1 gene. The carcinogenesis and cancer progression of tissue to be examined can be screened by detecting the production of the polypeptide. Although an antibody manufactured with the composition is specific to a human Nox1 polypeptide, the antibody can also be used in the screening of the carcinogenesis and cancer progression of non-human animals when having specificity to homologues of the polypeptide produced by the non-human animals. Thus, the composition for producing an antibody can also be used for producing an antibody for detecting a human cancer cell and an antibody for screening the carcinogenesis and cancer progression of mammals capable of producing human Nox1 polypeptide homologues.

The partial sequence of the amino acid sequence of SEQ ID NO: 2 is a sequence that comprises at least 8 or more consecutive amino acid residues contained in the amino acid sequence and refers to any of those serving as an epitope that induces the production of an antibody specific to the polypeptide comprising the amino acid sequence of SEQ ID NO: 2. When the partial sequence is used as an epitope, it can be used in combination with an appropriate carrier protein.

The polypeptide fragment of the present invention is any fragment of the polypeptides (1) and (2) and induces the production of an antibody specific to the polypeptide comprising the amino acid sequence of SEQ ID NO: 2.

The length of the peptide fragment is not particularly limited and however, is typically 20 to 200 amino acid residues, preferably 20 to 100 amino acid residues, more preferably 20 to 70 amino acid residues, in length.

In the present specification, the N-terminuses (amino terminuses) of the polypeptide, the homologue, and the polypeptide fragment (hereinafter, abbreviated to the polypeptide, if necessary) are indicated in the left, and the C terminuses (carboxyl terminuses) thereof are indicated in the right, in accordance with the standard notation. The polypeptide and so on, of the present invention can have a carboxyl group (—COOH), carboxylate (—COO—), amide (—CONH₂), or ester (—COOR) at the C terminus. Examples of the side chain R of this ester include: C₁ to C₆ alkyl groups such as methyl, ethyl, n-propyl, isopropyl, and n-butyl; C₃ to C₈ cycloalkyl groups such as cyclopentyl and cyclohexyl; C₆ to C₁₂ aryl groups such as phenyl and α-naphthyl; and alkyl groups such as phenyl-C₁ to -C₂ alkyl groups such as benzyl and phenethyl or α-naphthyl-C₁ to -C₂ alkyl groups such as α-naphthylmethyl.

The polypeptide and so on, of the present invention also includes: those in which an amino group of an N-terminal amino acid residue is protected with a protecting group such as a formyl group and an acetyl group; those in which an N-terminal glutamine residue generated in vivo by cleavage is converted to pyroglutamic acid; those in which a substituent (e.g., —OH, —SH, an amino group, an imidazole group, an indole group, or a guanidino group) on the side chain of an intramolecular amino acid is protected with a suitable protecting group; or a conjugated protein such as so-called glycoprotein bound with a sugar chain.

Moreover, the polypeptide and so on, of the present invention can be used as a physiologically acceptable inorganic or organic acid-addition salt. Examples of the inorganic acid-addition salt include salts of hydrochloric acid, phosphoric acid, hydrobromic acid, and sulfuric acid, and examples of the organic acid-addition salt include salts of acetic acid, formic acid, propionic acid, fumaric acid, maleic acid, succinic acid, tartaric acid, citric acid, malic acid, oxalic acid, benzoic acid, methanesulfonic acid, and benzenesulfonic acid.

Namely, the composition for producing an antibody of the present invention comprises the polypeptide, the homologue thereof, and their peptide fragments, or their acid-addition salts. The composition may optionally contain ingredients such as a vehicle, a diluent, and an adjuvant.

The present invention provides a method of producing an antibody specific to the polypeptide comprising the amino acid sequence of SEQ ID NO: 2, the homologue thereof, or the peptide fragment by use of the composition for producing an antibody.

An antibody manufactured by the method of the present invention is not particularly limited as long as it can be used according to the object of the present invention. Examples of the antibody include a human/mouse chimeric antibody, a humanized antibody, or a human antibody. In this context, the humanized antibody refers to any of those containing several percent of a mouse-derived antibody in the whole antibody, while the human antibody refers to an antibody derived 100% from a human. Meanwhile, the chimeric human antibody refers to any of those containing 10 to 20% of a mouse-derived antibody. The antibody may be any of polyclonal and monoclonal antibodies.

The monoclonal antibody of the present invention can be manufactured by the following procedures: at first, the composition for producing an antibody is administered to non-human mammals. A complete Freund's adjuvant or incomplete Freund's adjuvant may be administered for enhancing the ability of the mammals to produce antibodies. The administration is typically performed once every 2 to 6 weeks with a total of approximately 2 to 10 doses. Examples of the non-human mammals include monkeys, rabbits, dogs, guinea pigs, mice, rats, sheep, goats, and chickens, with mice and rats generally preferred.

An individual in which antibody production has been observed is selected from the non-human mammals immunized with the composition. Two to Five days after final immunization, antibody-producing cells contained in the spleen or lymph node are collected from the individual and fused with myeloma cells from animals of the same or different species to thereby create monoclonal antibody-producing hybridomas. Antibody titer in antiserum is measured by allowing a labeled protein to react with the antiserum and then measuring the activity of the labeling agent bound with the antibody. Cell fusion can be performed by a routine method such as the method of Kohler and Milstein (Nature, 256, 495, 1975). For example, polyethylene glycol (PEG) or a Sendai virus can be used as a fusion promoter.

Examples of the myeloma cells include non-human mammalian myeloma cells such as NS-1, P3U1, SP2/O, and AP-1, with P3U1 particularly preferred. A preferable ratio of the number of the antibody-producing cells (spleen cells) to the number of the myeloma cells, used in the fusion, is approximately 1:1 to 20. The spleen and myeloma cells can be fused by incubation at 20 to 40° C., preferably 30 to 37° C., for 1 to 10 minutes, with PEG (preferably PEG1000 to PEG6000) added at a concentration of approximately 10 to 80%.

The monoclonal antibody-producing hybridomas can be screened by a variety of methods. Examples of the methods include: a method that involves bringing a hybridoma culture supernatant into contact with a solid phase (e.g., a microplate) where an antigen is adsorbed either directly or in combination with a carrier and subsequently bringing a solution containing an anti-immunoglobulin antibody labeled with a radioactive substance, an enzyme, or the like, into contact therewith to detect the monoclonal antibody bound to the solid phase; and a method that involves bringing a hybridoma culture supernatant into contact with a solid phase where an anti-immunoglobulin antibody or the like is adsorbed and subsequently bringing a solution of a protein labeled with a radioactive substance, an enzyme, or the like, into contact therewith to detect the monoclonal antibody bound to the solid phase.

The monoclonal antibody-producing hybridomas can be selected by a routine method. For example, a medium for animal cells supplemented with HAT (hypoxanthine-aminopterin-thymidine), an RPMI 1640 medium containing 10 to 20% fetal bovine serum, a GIT medium containing 1 to 10% fetal bovine serum (Wako Pure Chemical Industries), or a serum-free medium for culturing hybridomas (SFM-101; Nissui Pharmaceutical) can be used. A culture temperature is 20 to 40° C., and a culture time is typically 5 days to 3 weeks. The culture can typically be performed under 5% carbonic acid gas. Antibody titer in the hybridoma culture supernatant can be measured in the same way as in the above-described measurement of antibody titer in antiserum.

The obtained monoclonal antibody can be separated and purified by a routine method such as: an immunoglobulin separation and purification method such as salting-out, alcohol precipitation, isoelectric precipitation, and electrophoresis; an absorption and desorption method with an ion exchanger (DEAE); an ultracentrifugation method; a gel filtration method; an antigen-bound solid phase; or a specific purification method performed by collecting only an antibody with a protein A active adsorbent and then dissociating the bond to obtain the antibody.

Alternatively, the polyclonal antibody of the present invention can be manufactured by immunizing non-human mammals with the polypeptide and so on serving as an immunizing antigen or a complex of the peptide fragment thereof with a carrier protein and then collecting an antibody-containing component such as serum therefrom, followed by antibody separation and purification.

The mixing ratio of the immunizing antigen to the carrier protein is determined so that the antibody can efficiently be raised against the hapten that has been crosslinked with the carrier and used in immunization. For example, approximately 0.1 to 20 parts by weight, preferably approximately 1 to 5 parts by weight, of bovine serum albumin, bovine thyroglobulin, or hemocyanin can be used with respect to 1 part by weight of the hapten. For example, glutaraldehyde, carbodiimide, maleimide-activated ester, and activated ester reagents containing a thiol or dithiopyridyl group can be employed in the coupling between the hapten and the carrier.

The complex antigen may be administered alone or in combination with a carrier or diluent, or alternatively, for enhancing the ability of the mammals to produce antibodies, with a complete Freund's adjuvant or incomplete Freund's adjuvant. The administration is typically performed once every 2 to 6 weeks with a total of approximately 3 to 10 doses. The polyclonal antibody is collected from the blood, ascites, yolk, or the like, of the immunized mammal. Polyclonal antibody titer in antiserum can be measured in the same way as in the above-described measurement of antibody titer in antiserum. The polyclonal antibody can be separated and purified in the same way as in the above-described separation and purification of the monoclonal antibody.

A diagnostic kit for cancer of the present invention comprises the polyclonal or monoclonal antibody thus obtained. The diagnostic kit optionally contains wells for immune reaction, a staining agent, an enzyme-labeled antibody for detection, a cleansing liquid, an antibody diluent, a sample diluent, an enzyme substrate, a diluent of an enzyme substrate solution, and additional reagents.

A pharmaceutical composition comprising the antibody of the present invention suppresses the expression of a specific protein coded for a Nox1 gene and as such, can be employed in the prevention, delayed progression, and treatment of carcinogenesis.

The antibody can be administered orally or parenterally, and the parenteral administration includes local administration to tissue.

When the pharmaceutical composition of the present invention is orally administered, pharmaceutical ingredients such as carriers widely used, for example, fillers, expanders, binders, disintegrants, disintegration inhibitors, buffers, tonicity agents, emulsifiers, dispersants, stabilizers, coating agents, surfactants, absorption promoters, humectants, wetting agents, adsorbents, lubricants, and excipients can be used. Additives such as coloring agents, preservatives, perfumes, flavoring agents, and sweetening agents may optionally be used.

Concrete examples of the pharmaceutical ingredients include: excipients such as milk sugar, white sugar, sodium chloride, grape sugar, urea, starch, calcium carbonate, kaoline, crystalline cellulose, and silicic acid; binders such as water, ethanol, simple syrup, grape sugar fluids, starch fluids, gelatin solutions, carboxymethylcellulose, shellac, methylcellulose, potassium phosphate, and polyvinylpyrrolidone; disintegrants such as dried starch, sodium alginate, agar powder, laminaran powder, sodium hydrogencarbonate, calcium carbonate, polyoxyethylene sorbitan fatty acid ester, sodium lauryl sulfate, stearic acid monoglyceride, starch, and milk sugar; disintegration inhibitors such as white sugar, stearic acid, cacao butter, and hydrogenated oil; absorption promoters such as quaternary ammonium salts and sodium lauryl sulfate; humectants such as glycerin and starch; adsorbents such as starch, milk sugar, kaolin, bentonite, and colloidal silicic acid; and lubricants such as purified talc and stearate. In addition, the technique of a drug delivery system known in the art is optionally adopted for each of the dosage forms to provide for the sustained release, local application (e.g., troches, buccals, and sublingual tablets), drug release control, conversion to an enteric coated form, conversion to a gastrolytic form, and so on, of the pharmaceutical composition.

When the pharmaceutical composition of the present invention is parenterally administered, the pharmaceutical composition can be used in forms such as administration by injection such as instillation, intravenous injection, hypodermic injection, and intramuscular injection, and endorectal administration with suppositories, for example, suppositories made of fats and oils and water-soluble suppositories. The pharmaceutical composition in such a form can be prepared with ease by a routine method using a typical carrier in the pharmaceutical field.

The pharmaceutical composition comprising the antibody of the present invention can be administered to, for example, humans or other mammals (e.g., rats, mice, guinea pigs, rabbits, sheep, pigs, cattle, horses, cats, dogs, and monkeys). The amount of the pharmaceutical composition administered appropriately differs depending on the conditions of objects to be administered, administration routes, and so on. For example, when orally administered, the pharmaceutical composition is generally administered at approximately 10 to 4000 mg, preferably approximately 20 to 2000 mg, more preferably approximately 50 to 500 mg, per day for an adult patient weighing 60 kg. When parenterally administered, it is preferred that the siRNA, for example in the form of injection, should be administered through the vein at a dose of approximately 10 to 2000 mg, preferably approximately 20 to 1000 mg, more preferably approximately 50 to 500 mg, per day for an adult patient weighing 60 kg although the dose differs depending on objects to be administered, the condition of liver cancer, and so on.

Cancer diagnosis can be performed by detecting and quantifying a Nox1 gene in biological tissue or bodily fluids by immunochemical assay comprising bringing the antibody of the present invention into contact with a biological sample.

When this immunochemical assay is conducted, the antibody of the present invention is held in a carrier. Examples of the carrier used in the assay include: gel particles such as agarose gels (e.g., Sepharose 4B and Sepharose 6B; Pharmacia Fine Chemicals), dextran gels (e.g., Sephadex G-75, Sephadex G-100, and Sephadex G-200; Pharmacia Fine Chemicals), polyacrylamide gels (Bio-Gel P-30, Bio-Gel P-60, and Bio-Gel P-100; BioRad Laboratories), and cellulose particles (Avicel; Asahi Kasei); ion-exchange cellulose such as diethylaminoethylcellulose and carboxymethylcellulose; physical adsorbents such as glass, silicone strips, and stainless steel-based resins; immunoassay plates (Nunc); and weakly basic anion-exchange resins (e.g., Amberlite IR-4B and DOWEX-3; Dow Chemical).

The antibody can be held in the carrier by a routine method such as cyanogen bromide and glutaraldehyde methods. In a more convenient way, the carrier may be adsorbed physically onto the surface of the antibody. Examples of a labeling agent to be bound to the antibody include radioactive isotopes, enzymes, fluorescent substances, and luminescent substances, with enzymes preferably used.

Any of those being stable and having high specific activity is preferable as the enzyme, and peroxidase, alkaline phosphatase, P-D-galactosidase, and glucose oxidase can be used, with peroxidase particularly preferred.

A sample to be tested in the immunochemical assay system of the present invention is a bodily fluid such as urine, serum, plasma, and spinal fluid, or an animal cell or a culture supernatant thereof. When the immunochemical assay of the present invention is conducted, a substance to be analyzed such as a Nox1 polypeptide to be measured is added to the antibody held in the carrier to perform antigen-antibody reaction. Then, the bound substance of an anti-Nox1 antibody and the labeling agent is further added thereto and reacted. The resulting reaction product is supplemented with the labeling agent (e.g., substrate of enzyme). The enzyme activity of the reaction product can be determined by measuring the absorbance or fluorescence intensity of a generated substance. These procedures are performed in advance on a standard solution of a known amount of the labeling agent (e.g., enzyme) to construct a standard curve that plots the relationship of absorbance or fluorescence intensity versus the amount of the Nox1 polypeptide or the like. This standard curve is compared with the absorbance or fluorescence intensity obtained from the substance to be analyzed (sample to be tested) comprising an unknown amount of the Nox1 polypeptide to measure the amount of the Nox1 polypeptide in the substance to be analyzed.

Moreover, the present invention provides a diagnostic method for cancer, characterized by detecting a polynucleotide comprising the nucleotide sequence of SEQ ID NO: 1 or a fragment thereof. The polynucleotide or the fragment thereof can be detected by a routine method utilizing polymerase chain reaction (PCR) or real-time quantitative polymerase chain reaction.

The polymerase chain reaction or the real-time quantitative polymerase chain reaction uses a sense strand fragment corresponding to the nucleotide sequence of SEQ ID NO: 1 as a forward primer and an antisense strand fragment corresponding to the nucleotide sequence of SEQ ID NO: 1 as a reverse primer. The length of the forward primer dose not have to be particularly limited and however, is typically 14 to 60 bases. Similarly, although the length of the reverse primer dose not have to be particularly limited, it is usually preferred that the length should be 14 to 60 bases. Examples of the forward and reverse primers used in the diagnostic method for cancer of the present invention include the followings:

(SEQ ID NO: 5)

the forward primer of 5′-GGAGCAGGAATTGGGGTCAC-3′;
and

(SEQ ID NO: 6)

the reverse primer of 5′-TTGCTGTCCCATCCGGTGAG-3′.

When the detection is performed by the real-time quantitative polymerase chain reaction, examples of forward and reverse primers and a TaqMan probe used therein include the followings:

the forward primer of

(SEQ ID NO: 7)

5′-CCACTGTAGGCGCCCTAAGTT-3′;
the reverse primer of

(SEQ ID NO: 8)

5′-AAGAATGACCGGTGCAAGGA-3′;
and
the TaqMan probe of

(SEQ ID NO: 9)

5′-AAGGGCATCCCCCTGAGTCTTGGAA-3′.

In the present invention, siRNA or siRNA corresponding to a polynucleotide comprising the nucleotide sequence of SEQ ID NO: 1 or a fragment thereof suppresses Nox1 gene expression and reduces Nox1 polypeptide production. Thus, the use of the siRNA or oligonucleotide will allow the inhibition of cell carcinogenesis or cancer progression induced by a mutant Ras gene.

The siRNA or oligonucleotide of the present invention refers to a nucleotide or an oligonucleotide complementary to a polynucleotide comprising a consecutive nucleotide sequence of SEQ ID NO: 1 or a fragment thereof.

The siRNA of the present invention may correspond to a polynucleotide comprising a nucleotide sequence at positions from 71 to 1615 of SEQ ID NO: 1 or a fragment thereof. The length of the siRNA is less than 100 bp and is preferably 8 to 99 bp, particularly preferably 10 to 30 bp. Moreover, preferable examples of the ssi include the followings:

siRNAs for human Nox1 of

(SEQ ID NO: 10)

5′-GCGTGGCTTCAGCATGGAATTCAAGAGATTCCATGCTGAAGCCACGC
TTTTTTGGAAA-3′;
and

(SEQ ID NO: 11)

5′-GGGCTTTCGAACAACAATATTCAAGAGATATTGTTGTTCGAAAGCCC
TTTTTTGGAAA-3′;
and
siRNAs for rat Noxi of

(SEQ ID NO: 12)

5′-GTTATGAGAAGTCTGACAAGTTCAAGAGACTTGTCAGACTTCTCATA
ATTTTTTGGAAA-3′;

(SEQ ID NO: 13)

5′-GATTCTTGGCTAAATCCCATTCAAGAGATGGGATTTAGCCAAGAATC
TTTTTTGGAAA-3′;
and

(SEQ ID NO: 14)

5′-GGACATTTGAACAACAGCATTGAAGAGATGCTGTTGTTCAAATGTCC
TTTTTTGGAAA-3′.

The siRNA of the present invention can be used by directly administrating the siRNA to a patient to be treated; by infecting the siRNA into a cell taken out of a patient to be treated and then returning the cell to the patient; or by administering an expression vector incorporating the siRNA therein to a patient to be treated and expressing the siRNA.

Thus, the present invention provides a pharmaceutical composition for cancer therapy, comprising: siRNA corresponding to a polynucleotide comprising the nucleotide sequence of SEQ ID NO: 1 or a fragment thereof; and siRNA corresponding to a polynucleotide comprising a nucleotide sequence at positions from 71 to 1615 of SEQ ID NO: 1 or a fragment thereof.

The pharmaceutical composition can be manufactured as the foregoing pharmaceutical composition comprising the antibody, by a routine method utilizing a pharmaceutical material conventionally used. The pharmaceutical composition comprising the siRNA of the present invention can be administered to, for example, humans or other mammals (e.g., rats, mice, guinea pigs, rabbits, sheep, pigs, cattle, horses, cats, dogs, and monkeys). The amount of the pharmaceutical composition administered appropriately differs depending on the conditions of objects to be administered, administration routes, and so on. For example, when orally administered, the pharmaceutical composition is generally administered at approximately 10 to 4000 mg, preferably approximately 20 to 2000 mg, more preferably approximately 50 to 500 mg, per day for an adult patient weighing 60 kg. When parenterally administered, it is preferred that the siRNA, for example in the form of injection, should be administered through the vein at a dose of approximately 10 to 2000 mg, preferably approximately 20 to 1000 mg, more preferably approximately 50 to 500 mg, per day for an adult patient weighing 60 kg although the dose differs depending on objects to be administered, the condition of cancer to be treated, and so on.

Moreover, the present invention provides a pharmaceutical composition for cancer therapy, comprising a cell transformed with siRNA corresponding to a polynucleotide comprising the nucleotide sequence of SEQ ID NO: 1 or a fragment thereof; and siRNA corresponding to a polynucleotide comprising a nucleotide sequence at positions from 71 to 1615 of SEQ ID NO: 1 or a fragment thereof.

In this context, the cell to be transformed may be a cell donated from a third party or a cell taken out of a patient to be treated. Alternatively, the transformed cell can be used as a cell for cancer therapy.

Moreover, the present invention provides an RNA molecule consisting of a partial sequence of the nucleotide sequence of SEQ ID NO: 1, or an RNA molecule consisting of a mutant nucleotide sequence of the partial sequence with the addition, deletion, or substitution of at least one base, and suppressing Nox1 polypeptide expression in a human cell. The RNA molecule (siRNA) can be designed on the basis of the sequence of the polynucleotide of the present invention according to a method known in the art (e.g., Nature, Vol. 411, pp. 494, 2001).

When the RNA molecule of the present invention is used in vivo or in vitro, it is used in the form of a double-stranded RNA molecule that consists of the RNA molecule and its complementary RNA. In this case, it is preferred that they should be treated so that the double-stranded RNA molecule is not degraded in a cell or is not dissociated into single strands. The treatment is performed by, for example, a method that adds a hydroxyl group to the 3′ end of the RNA molecule, forms chemical bonds at both ends of the double strands through thiophosphoryl groups, or induces a chemical bond between both strands by ultraviolet radiation or through a bifunctional group such as bis(2-chloromethyl)amine, 4-thiouracil, or psoralen. The RNA molecule of the present invention suppresses Nox1 gene expression accompanying transformation with a mutant Ras gene and as such, can be used as a pharmaceutical composition for the prevention of cell carcinogenesis, the inhibition of cancer progression, and cancer therapy.

Moreover, the present invention provides a screening method for a cancer cell growth inhibitor targeted for a Nox1 gene. The screening method comprises bringing a cell having a mutant Ras gene or a normal cell transfected with a Nox1 gene into contact with a substance to be screened and then detecting the expression of the Nox1 gene and the inactivation of its enzyme activity. Subsequently, the transformed cell is cultured together with the substance to be screened to examine its ability to inhibit growth.

The influence of a compound to be screened on Nox1 gene expression can be examined by detecting an increase or decrease in mRNA expression by real-time quantitative polymerase chain reaction; by detecting a polypeptide or a peptide fragment thereof coded for the Nox1 gene with an antibody; or by observing morphological changes in the transformed cell.

The cell having a mutant Ras gene that can be used in the screening method, is not particularly limited, and examples thereof include H-Ras-NIH3T3 cells, K-Ras-NRK cells, and pancreatic cancer cells. For example, pEGFP-C1 (control) and pEGFP-C1-Nox1 can be used as a vector used in the introduction of the Nox1 gene, while for example, pSilencer can be used as an siRNA-expressing vector.

A cell line highly expressing Nox1 can be established by stably transfecting the pEGF-C1-Nox1 vector into an NIH3T3 or NRK cell by use of a Lipofectamine method. Alternatively, a pancreatic cancer cell having a mutant Ras gene can be utilized. It is preferred to use any of those separated and established from a human pancreatic cancer patient as the pancreatic cancer cell. These cells are cultured in a standard culture solution (Dulbecco's modified Eagle medium (DMEM) containing 10% fetal bovine serum (FBS)) in the presence of 5% CO₂. High-throughput screening can be conducted by culturing the cells in a multi-plate (96 or 48 wells), to which the substance to be screened is then added.

### EXAMPLE 1

### Confirmation of Nox1 Gene Expression Elevated by Mutant Ras Gene

Because Nox1 gene overexpression in NIH3T3 cells elevates superoxide generation and cell growth (1 and 2), the present inventors assumed that Nox1 is associated with the phenotypic expression of particular oncogenes. Therefore, cells transformed with K-Ras Val12 oncogene were used to confirm Nox1 gene expression elevated by Ras oncogene.

At first, rat kidney (NRK) cells and K-Ras-NRK cells (Kirstein murine sarcoma virus-transformed NRK cells) were prepared. The K-Ras-NRK cells were purchased from ATCC.

The presence or absence of Nox1 expression in the rat kidney (NRK) cells and the K-Ras-NRK cells was initially detected by real-time quantitative polymerase chain reaction. The real-time quantitative polymerase chain reaction used a forward primer of SEQ ID NO: 15, a reverse primer of SEQ ID NO: 16, and a TaqMan MGB probe of SEQ ID NO: 17.

Forward Primer:

(SEQ ID NO: 15)

5′-GGTCACTCCCTTTGCTTCCA-3′
Reverse Primer:

(SEQ ID NO: 16)

5′-GGCAAAGGCACCTGTCTCTCT-3′
TaqMan MGB Probe:

(SEQ ID NO: 17)

5′-TCCAGTAGAAATAGATCTTT-3′

The real-time quantitative polymerase chain reaction was performed using ABI Prism 7700 (Applied Biosystems) under reaction conditions set to defaults. Analytical values were standardized using rRNA 18S.

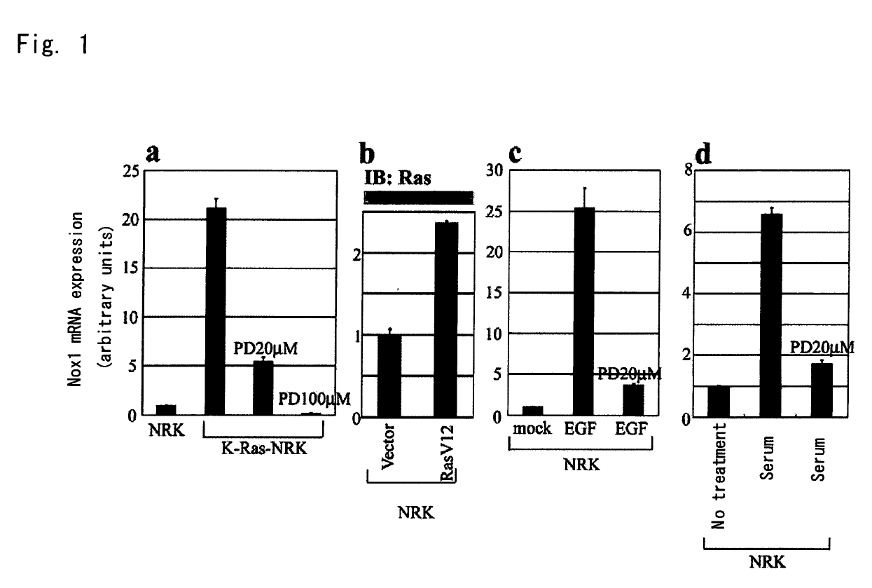

As a result of gene expression analysis by the real-time quantitative polymerase chain reaction, Nox1 gene expression was elevated in the K-Ras-NRK cells transformed with the K-Ras oncogene, as shown in the graph of FIG. 1(a).

### EXAMPLE 2

### Confirmation of Nox1 Gene Expression Elevated by Transient Transfection with Mutant Ras Gene

For analyzing the influence of transient transfection with mutant Ras gene, pcDNA3.1 vectors (blank vectors) and pcDNA3.1 carrying Ras Val12 vectors were used and transiently transfected into NRK cells. Forty-eight hours after the transfection, real-time quantitative polymerase chain reaction was conducted in the same way as in Example 1 in order to detect Nox1 expression. As a result, Nox1 gene expression was also elevated in the NRK cells transiently transfected with the H-Ras Val12, as compared with Nox1 gene expression in the control vector used, as shown in FIG. 1(b). The expression of the transfected Ras V-12 was confirmed by immunoblotting (IB) using a rabbit anti-Ras antibody.

For performing the immunoblotting, 1×10⁵ sample cells were solubilized with 2× sample buffers (0.1 M Tris-Cl (pH 6.8), 20% glycerol, 4% SDS, 3.1% DTT, and 0.001% BPB), and the polypeptides were separated by SDS gel electrophoresis and subsequently analyzed by the immunoblotting.

The proteins were electrically transferred to a nitrocellulose membrane in a transfer buffer (25 mM Tris-Cl (pH 8.3), 92 mM glycine, and 20% methanol) and reacted with an anti-Ras antibody (primary antibody) and an HRP-conjugated anti-rabbit-IgG antibody, followed by detection by a chemiluminescence (ECL) method.

As described above, the results of Examples 1 and 2 indicated that Nox1 gene expression is elevated by the action of mutant Ras oncogene.

NIH3T3 cells cultured overnight under serum-free conditions were treated mitogeniclally with serum (30%) or 50 ng/ml epidermal growth factor (EGF) and then analyzed by real-time quantitative polymerase chain reaction. As a result, Nox1 gene expression was increased to 6 to 20 times within 12 hours from the initiation of the culture, as shown in FIGS. 1(c) and (d). That is, Nox1 gene expression is associated with factors that promote cell growth. This is consistent with the reports saying that both platelet-derived growth factor (PDGF) and angiotensin II induce superoxide formation and Nox1 gene expression in vascular smooth muscle cells (1 and 4).

### EXAMPLE 3

### Analysis of Signaling Pathway of Nox1 Gene Expression Caused by Mutant Ras Gene

A series of experiments were performed for analyzing the downstream region of a Ras signaling pathway of Nox1 gene expression elevated by mutant Ras gene. In Examples 1 and 2, the cells were treated for 12 hours with PD98059 (PD: 20 μM, 100 μM), an MAPKK inhibitor, to analyze Nox1 expression in the K-Ras-NRK cells and serum- and EGF-induced Nox1 expression by real-time quantitative polymerase chain reaction.

As a result, Nox1 expression in the K-Ras-NRK cells was inhibited by the PD98059 concentration-dependently (FIG. 1(a)). Moreover, serum- and EGF-induced Nox1 expression was respectively inhibited by 20 μM PD98059 (FIGS. 1(c) and (d)).

On the other hand, wortmannin (100 nM), a PI3 kinase inhibitor, did not inhibit an increase in Nox1 expression in the H-Ras-NIH3T3 cells in a control experiment (data not shown). These results indicated that the signals of mutant Ras gene and growth factors thereof induce Nox1 expression, by pathways not via PI3K but via Ras-MAPKK-MAPK.

### EXAMPLE 4

### Involvement of Nox1 Gene in Transformation with Mutant Ras Gene

Oligonucleotides 1-S and 1-AS were annealed and subcloned into a pSilencer hygro vector with an H1-promoter (Ambion) to yield RNAi(1). Likewise, oligonucleotides 3-S and 3-AS were annealed and subcloned into a pSilencer hygro vector with an H1-promoter (Ambion) to yield RNAi(3). In addition, oligonucleotides 5-S and 5-AS were annealed and subcloned into a pSilencer hygro vector with an H1-promoter (Ambion) to yield RNAi(5). A pSilencer hygro Negative Control plasmid (Ambion) was used as a control vector.

The RNAi(1) is an siRNA construction targeted for positions from 223 to 241 of SEQ ID NO: 3. The RNAi(3) is an siRNA construction targeted for positions from 578 to 596 of SEQ ID NO: 3. The RNAi(5) is an siRNA construction targeted for positions from 1224 to 1242 of SEQ ID NO: 3.

The sequence of each oligonucleotide is as follows:

| | |
|---|---|
| 1-S: | |
| 5′-GATCCCGTTATGAGAAGTCTGACAAGTTCAA | (SEQ ID NO: 18) |
| GAGACTTGTCAGACTTCTCATAATTTTTTGGAA | |
| A-3′ | |
| 1-AS: | |
| 5′-AGCTTTTCCAAAAAATTATGAGAAGTCTGAC | (SEQ ID NO: 19) |
| AAGTCTCTTGAACTTGTCAGACTTCTCATAACG | |
| G-3′ | |
| 3-S: | |
| 5′-GATCCCGATTCTTGGCTAAATCCCATTCAAG | (SEQ ID NO: 20) |
| AGATGGGATTTAGCCAAGAATCTTTTTTGGAA | |
| A-3′ | |
| 3-AS: | |
| 5′-AGCTTTTCCAAAAAAGATTCTTGGCTAAATC | (SEQ ID NO: 21) |
| CCATCTCTTGAATGGGATTTAGCCAAGAATCG | |
| G-3′ | |
| 5-S: | |
| 5′-GATCCCGGACATTTGAACAACAGCATTCAAG | (SEQ ID NO: 22) |
| AGATGCTGTTGTTCAAATGTCCTTTTTTGGAA | |
| A-3′ | |
| 5-AS: | |
| 5′-AGCTTTTCCAAAAAAGGACATTTGAACAACA | (SEQ ID NO: 23) |
| GCATCTCTTGAATGCTGTTGTTCAAATGTCCG | |
| G-3′ | |

4 μg each of the RNAi(1), RNAi(3), RNAi(5), and pSilencer hygro Negative Control plasmid was transfected respectively into 1×10⁶ K-Ras-NRK cells using Lipofectamine (Gibco-BRL). The transfected cells were cultured for selection at 37° C. for 2 to 3 weeks in a DMEM medium supplemented with 10% fetal bovine serum (FBS) and 400 μg/ml hygromycin B under a 5% CO₂ moisture environment. After the culture, surviving colonies were isolated.

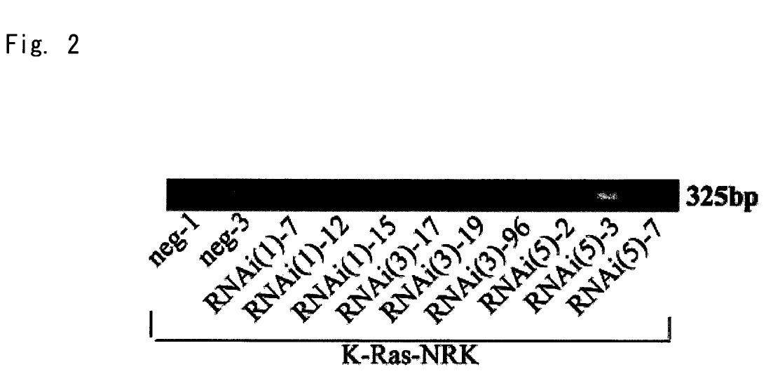

Three clones of the cell lines stably transfected with each of the RNAi(1), RNAi(3), and RNAi(5) (K-Ras-NRK/RNAi(1)-7, K-Ras-NRK/RNAi(1)-12, and K-Ras-NRK/RNAi(1)-15; K-Ras-NRK/RNAi(3)-17, K-Ras-NRK/RNAi(3)-19, and K-Ras-NRK/RNAi(3)-96; and K-Ras-NRK/RNAi(5)-2, K-Ras-NRK/RNAi(5)-3, and K-Ras-NRK/RNAi(5)-7) as well as two clones of the cells stably transfected with the pSilencer hygro Negative Control plasmid (K-Ras-NRK/neg-1 and K-Ras-NRK/neg-2) were selected. The transfection of these constructions was confirmed by PCR using M13F and 3.0Rev as primers (FIG. 2). PCR temperature conditions were set to 94° C. for 2 minutes, 30 cycles of (94° C. for 1 minute, 60° C. for 1 minute, and 72° C. for 1 minute), followed by 72° C. for 10 minutes. A thermal cycler used was Takara Thermal Cycler SP (Takara Shuzo Company Limited).

(Primer Sequence)

(SEQ ID NO: 24)

| | |
|---|---|
| M13F: | 5′-GTTTTCCCAGTCACGAC-3′ |

(SEQ ID NO: 25)

| | |
|---|---|
| 3.0Rev: | 5′-GAGTTAGCTCACTCATTAGGC-3′ |

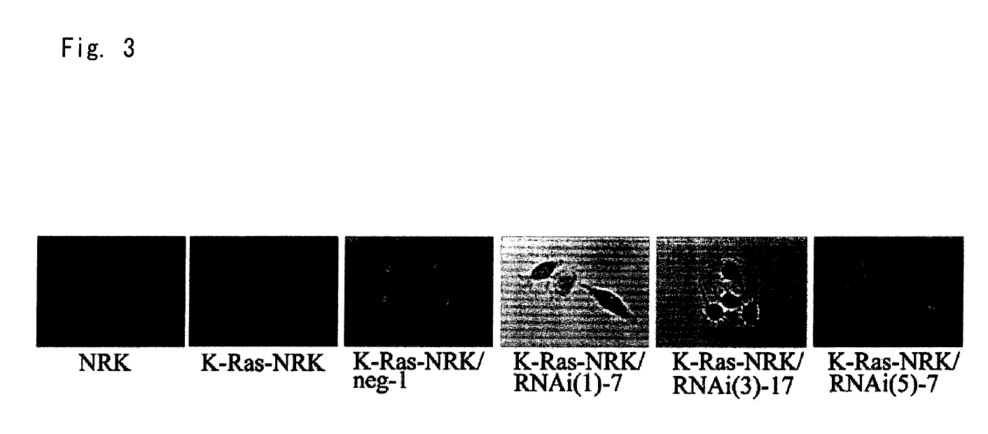

Next, celluler morphological changes in which Nox1 gene was involved were compared among the cells. As a result, as shown in FIG. 3, the K-Ras-NRK cells transfected with the RNAi(1) to RNAi(3) were extended, whereas the K-Ras-NRK cells transfected with the pSilencer hygro Negative Control plasmid morphologically remained round as the cells observed before introduction.

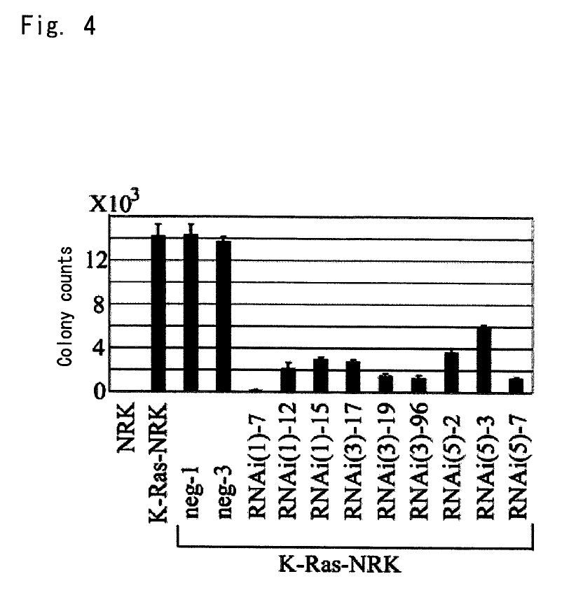

Subsequently, soft agar culture assay was performed in order to examine the transformation of the cells by observing anchorage-independent growth capacity. An agarose layer containing 0.53% agar and nutrients necessary for cell growth was placed and solidified in a culture dish of 6 cm in diameter, on which the cells suspended in a DMEM containing 0.3% agar and 10% FBS were then piled up at a final concentration of 1.5×10⁴ cells/culture dish. Subsequently, the cells were cultured at 37° C. under a 5% CO₂ moisture environment to observe the appearance of cell colonies over 3 weeks. The adhesion-dependent cell growth of the cell lines exhibited by the soft agar culture was measured, and +/−average standard deviation (s.e.m.) from three measurements was shown in FIG. 4. As a result, the formation of many colonies was observed in the K-Ras-NRK cells and the transfected cells with the pSilencer hygro Negative Control plasmid (neg-1 and neg-2), whereas colony formation was remarkably inhibited in the transfection with the RNAi(1) to RNAi(5).

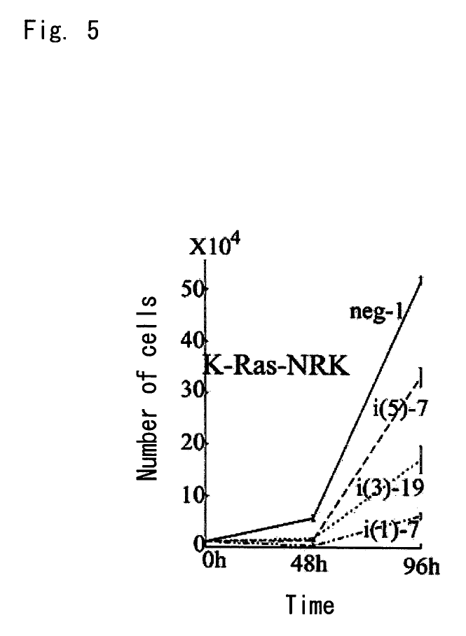

In addition, 10⁴ cells each of the K-Ras-NRK/neg-1 (neg-1), K-Ras-NRK/RNAi(1)-7 (i(1)-7), K-Ras-NRK/RNAi(3)-19 (i(3)-19), and K-Ras-NRK/i(5)-7 (i(5)-7) cells were transferred from the culture dish to a culture solution and then liquid-cultured for 6 days in a standard culture solution (DMEM containing 10% FBS) in the presence of 5% CO₂. Subsequently, the number of the cells in the liquid medium was measured, and its growth curve was shown in FIG. 5. As a result, each growth rate of the K-Ras-NRK/RNAi(1)-7, K-Ras-NRK/RNAi(3)-19, and K-Ras-NRK/RNAi(5)-7 cells was also decreased in the liquid culture, and by contrast, inhibitory effect on growth was not observed in the K-Ras-NRK/neg-1.

### EXAMPLE 5

### Confirmation of Superoxide Production by NADPH Oxidase Coded for Nox1 Gene

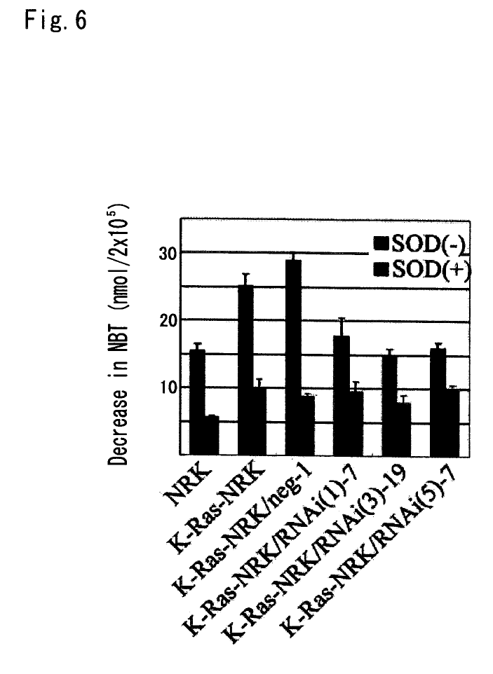

The present inventors measured superoxide production by NBT reduction analysis using the transformed cells of Example 4 in order to evaluate the role of Nox1 in superoxide production induced by mutant Ras. In the present example, the method of Suh et al. was used in the NBT (nitroblue tetrazolium) analysis (1). Namely, 0.2 ml of a Hanks solution containing 0.25% NBT was prepared with or without 40 units of superoxide dismutase (SOD), a reactive oxygen-digesting enzyme, in which 2×10⁵ cells were in turn suspended and treated at 37° C. for 8 minutes. The treated cells were separated by low-speed centrifugation and solubilized by the addition of 0.5 ml of pyridine. Absorbance at 510 nm was measured to quantify a decrease in NBT (extinction coefficient of 11,000/M/cm). The obtained data was shown in FIG. 6 with +/−s.e.m. from 3 measurements.

As a result of the analysis, the K-Ras-NRK and K-Ras-NRK/neg-1 increased NBT reduction, which was inhibited by the superoxide dismutase treatment, as compared with the NRK. By contrast, the K-Ras-NRK/RNAi(1)-7, K-Ras-NRK/RNAi(3)-19, and K-Ras-NRK/RNAi(5)-7 decreased the ROS production-stimulating effect of K-Ras oncogene to the level of the NRK cells (FIG. 6), thereby indicating that Nox1 is involved in an increase in superoxide production in mutant Ras-transformed cells.

### EXAMPLE 6

### Confirmation of Nox1 Expression Decreased by RNAi(1), RNAi(3), and RNAi(5)

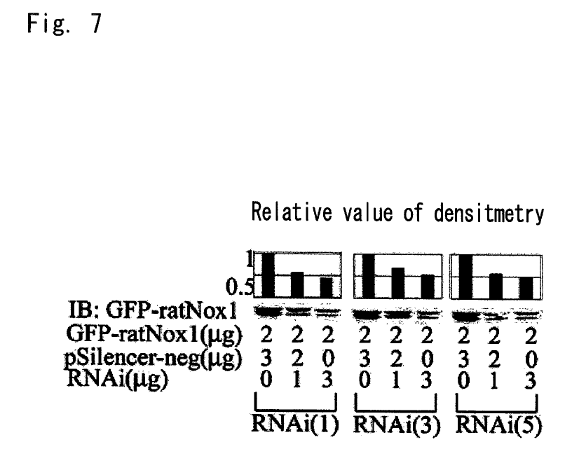

The present inventors conducted analysis in the following procedures in order to confirm Nox1 expression decreased by the RNAi(1), RNAi(3), and RNAi(5): GFP-rat Nox1 was subjected to cotransfection with each of the RNAi(1) to (5) and subsequently immunoblotting analysis was conducted in the same way as in Example 2 in order to evaluate the inhibitory effect of the RNAi(1), RNAi(3), and RNAi(5) on GFP-rat Nox1 expression. As shown in the left panel of FIG. 7, it was revealed that an expected GFP-rat Nox1 fusion polypeptide is produced, and that the coexpression of the RNAi(1), RNAi (3), and RNAi(5) suppresses the production of a GFP-rat Nox1 fusion protein dependently on a dose of these vectors. GFP-Nox1 expression shown in the left panel of FIG. 7 was quantitatively determined by immunoblotting using an anti-GFP antibody. Numerals in the left panel of FIG. 7 denote the amount (μg) of DNA transfected. Both RNAi(1) and RNAi(5) are targeted for rat Nox1. The same approach as in the left panel of FIG. 7 was used to show in the right panel of FIG. 7 that the RNAi(1) and RNAi(5) do not inhibit human Nox1, that is, the RNAi constructions have high specificity to their target gene.

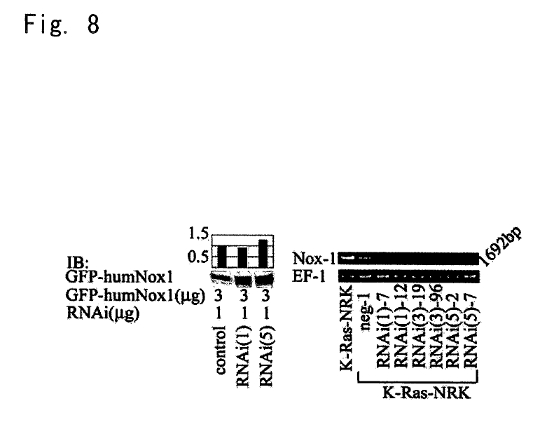

Moreover, RT-PCR demonstrated that the mRNA expression of endogenous Nox1 is certainly inhibited in each of the K-Ras-NRK/RNAi(1)-7, K-Ras-NRK/RNAi(1)-12, K-Ras-NRK/RNAi(3)-19, K-Ras-NRK/RNAi(3)-96, K-Ras-NRK/RNAi(5)-2, K-Ras-NRK/RNAi(5)-7 cells, as compared to the K-Ras-NRK and K-Ras-NRK/neg-1 cells. The result is shown in FIG. 8.

Sequences of PCR Primers used in Example 6

(SEQ ID NO: 26)

Forward Primer: 5′-ATGGGAAACTGGCTGGTTA-3′

(SEQ ID NO: 27)

Reverse Primer: 5′-TCAGAACGTTTCTTTGTTGAA-3′

The results of the present Example indicate that an increase in the expression rate of Nox1 gene and transformation with mutant Ras are associated with each other, as well as morphological changes caused by the constitutional changes of cytoskeletons and adhesive proteins.

### EXAMPLE 7

### Influence of Reactivation of Nox1 Once Inhibited by siRNA on Cell

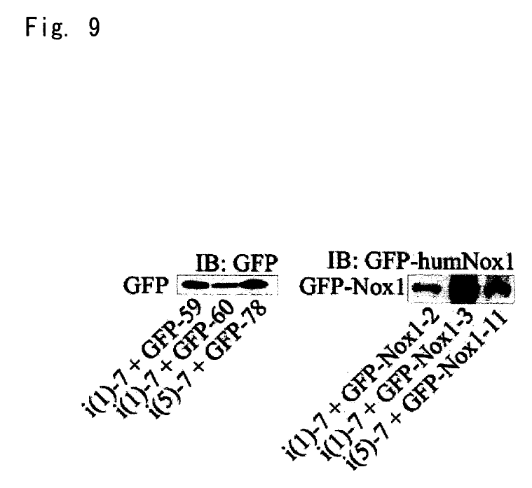

pEGFP-C1 (GFP) and pEGFP-human Nox1 (GFP-Nox1) were separately transfected into the K-Ras-NRK/RNAi(1)-7 and K-Ras-NRK/RNAi(5)-7 by the same approach as in Example 4, then immunoblotting was conducted by the same approach as in Example 6 (FIG. 9). As shown in Example 6 and the right panel of FIG. 7, neither RNAi(1) nor RNAi(5) inhibits human Nox1 expression. Therefore, even if the pEGFP-human Nox1 is transfected into the K-Ras-NRK/RNAi(1)-7 and K-Ras-NRK/RNAi(5)-7 cells, this human Nox1 is not inhibited by siRNA.

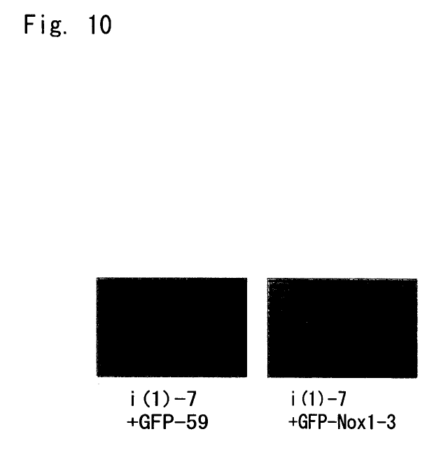

As shown in FIG. 10, the K-Ras-NRK/RNAi(1)-7 cells could not derepress siRNA and were oblate only by introducing the control vector therein (i(1)-7+GFP), whereas they morphologically returned to the spherical shape close to the original K-Ras-NRK cells by transfecting the pEGFP-human Nox1 therein (i(1)-7+GFP-Nox1-3) (FIG. 10).

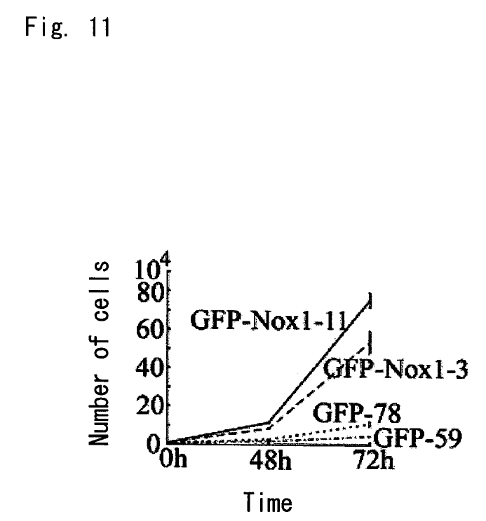

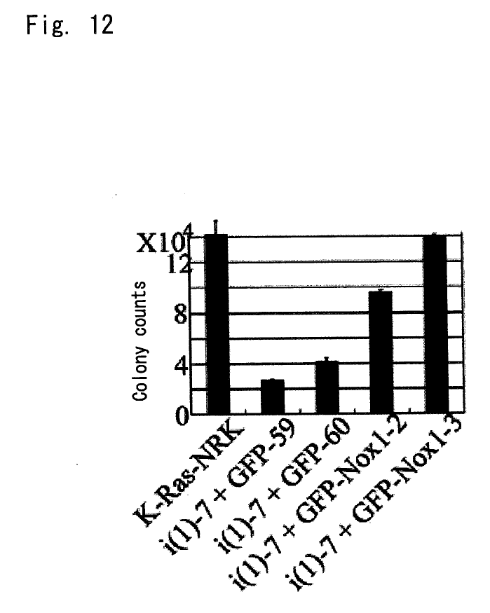

Growth curves obtained by the same approach as in Example 4 also exhibited accelerated growth in the pEGFP-human Nox1-incorporated K-Ras-NRK/RNAi(1)-7 (GFP-Nox1-3) and pEGFP-human Nox1-incorporated K-Ras-NRK/RNAi(5)-7 (GFP-Nox1-1), as compared with the control vector-incorporated K-Ras-NRK/RNAi(1)-7 (GFP-59) and the control vector-incorporated K-Ras-NRK/RNAi(5)-7 (GFP-78) (FIG. 11). Moreover, the soft agar culture assay in the same way as in Example 4 also demonstrated that colony formation in the pEGFP-human Nox1-incorporated K-Ras-NRK/RNAi(1)-7 (GFP-Nox1-2 and GFP-Nox1-3) recovered to the same level as that in the original K-Ras-NRK cells, as compared with the control vector-incorporated K-Ras-NRK/RNAi(1)-7 (i(1)-7+GFP-59 and i(1)-7+GFP-60) (FIG. 12).

### EXAMPLE 8

### Influence of Nox1 Gene Expression on Transformation with Mutant Ras

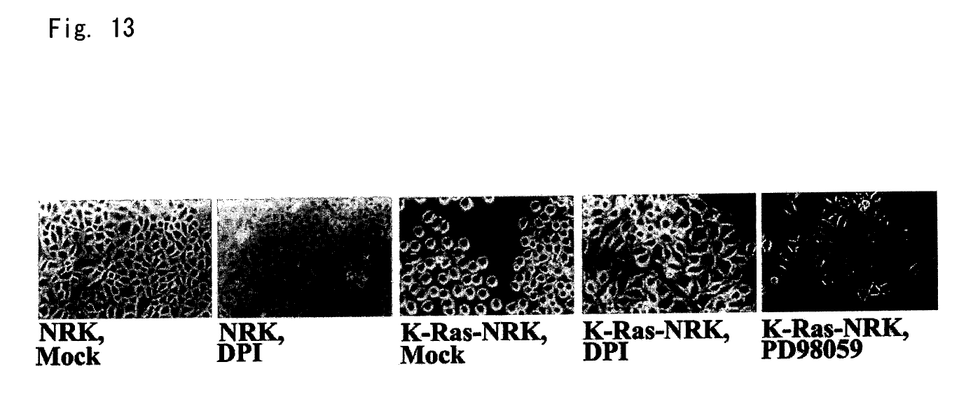

Diphenylene iodonium (DPI), a Nox1 inhibitor, was used to examine the influence of Nox1 gene expression on the morphologies of the cells transformed with mutant Ras. Namely, K-Ras-NRK cells and NRK cells were cultured overnight at 37° C. in a medium containing 20 μM DPI or 30 μM PD98059 under a 5% CO₂-containing moisture environment to observe morphological changes. Photographs documenting the cell morphologies are shown in FIG. 13.

As shown in FIG. 13, the K-Ras-NRK cells transiently assumed a flat shape that was close to the shape of the NRK cells, when the K-Ras-NRK cells were treated overnight with flavoprotein inhibitor DPI or 10 mM antioxidant n-acetyl cysteine (data not shown). As further shown in FIG. 13, such morphological changes were also observed in the K-Ras-NRK cells treated with the PD98059. Thus, these results indicated that an increase in Nox1 gene expression is essential to Ras-MAPKK-MAPK pathway-promoted transformation with mutant Ras.

### EXAMPLE 9

### Influence of siRNA on Tumor Formation Caused by Mutant Ras

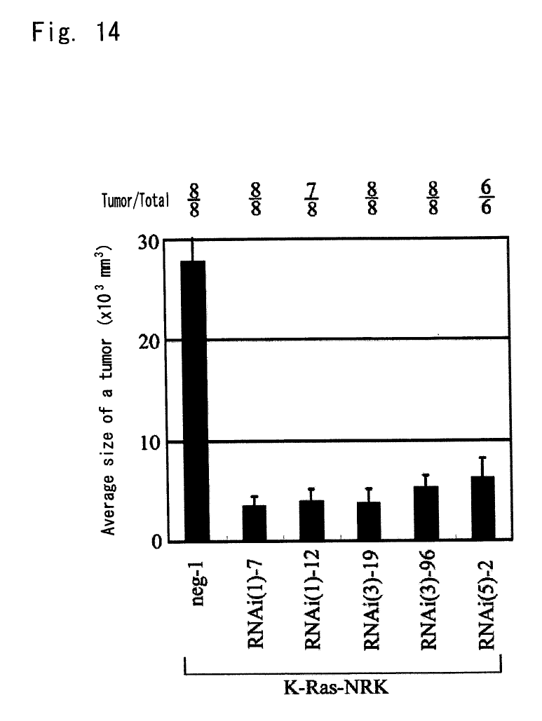

The K-Ras-NRK/neg-1, K-Ras-NRK/RNAi(1)-7, K-Ras-NRK/RNAi(1)-12, K-Ras-NRK/RNAi(3)-19, K-Ras-NRK/RNAi(3)-96, and K-Ras-NRK/RNAi(5)-2 cells were separately transplanted into athymic mice to observe tumor formation. Namely, 10⁶ cells each of the cells were suspended in 0.2 ml PBS and then subcutaneously transplanted into the nude mice (athymic: Nu/Nu). Then, tumor formation in all of the nude mice was observed over 1 month, while the volume of the formed tumor was measured. The result was shown in FIG. 14. In FIG. 14, the solidly shaded bars denote the volume of tumor; the error bars denote s.e.m.; and the fractions denote the proportion of tumor-bearing mice to all the mice used (tumor/total).

As shown in FIG. 14, the K-Ras-NRK/neg-1 formed active tumor within 2 weeks. By contrast, the K-Ras-NRK/RNAi(1)-7, K-Ras-NRK/RNAi(1)-12, K-Ras-NRK/RNAi(3)-19, and K-Ras-NRK/RNAi(3)-96 remarkably suppressed tumor growth. When histologically observed by anatomy, the tumor formed in the K-Ras-NRK/neg-1 resulted in increased blood vessels. This is consistent with the possibility that Nox1 gene induces angiogenesis caused by increase in vascular endothelial growth factor production (1). The small tumor formed in the K-Ras-NRK/RNAi(1)-7, K-Ras-NRK/RNAi(1)-12, K-Ras-NRK/RNAi(3)-19, and K-Ras-NRK/RNAi(3)-96 exhibited signs of necrosis in most of the cells (data not shown).

As seen from these experimental results, mutant Ras (oncogene) elevates mitogenic oxidase Nox1 via MAPKK-MAPK pathways, whereas Nox1 gene expression is essential to transformation of cells, cancer formation, and its progression caused by the Ras. Namely, the studies conducted by the present inventors revealed a molecular mechanism in which a Nox1 polypeptide plays an essential role as a redox signal molecule in transformation with mutant Ras.

Nox family proteins Nox1 to 5, Gp91phox homologues, were identified from non-phagocytic cells and reported to respectively have specific functions in various cell processes (1 and 5 to 9). Among the Nox family, the Nox1 gene is unique in that it codes for transmembrane oxidase that mediates the mitogenic signals from growth factors (1 and 4) and oncogenes.

Considering the involvement of ROS in the promotion of tumor growth and the possibility of ROS production for inducing ROS-producing enzymes in malignant cancer cells (7), Nox1 gene and its polypeptides are probably capable of suppressing cancer progression and serving as target molecules that allow the cancer therapy.

The SEQ ID NOs in the sequence listing of the present specification represent the following sequences:

SEQ ID NO: 1 represents the nucleotide sequence of mRNA transcribed from a human Nox1 gene or cDNA;

SEQ ID NO: 2 represents the amino acid sequence of a polypeptide coded by the human Nox1 gene;

SEQ ID NO: 3 represents the nucleotide sequence of mRNA transcribed from a rat Nox1 gene or cDNA;

SEQ ID NO: 4 represents the amino acid sequence of a polypeptide coded by the rat Nox1 gene;

SEQ ID NO: 5 represents the nucleotide sequence of a forward primer corresponding to a Nox1 gene used in a diagnostic method for human cancer;

SEQ ID NO: 6 represents the nucleotide sequence of a reverse primer corresponding to a Nox1 gene used in a diagnostic method for human cancer;

SEQ ID NO: 7 represents the sequence of a forward primer used in the detection of a human Nox1 gene by real-time quantitative polymerase chain reaction;

SEQ ID NO: 8 represents the sequence of a reverse primer used in the detection of a human Nox1 gene by real-time quantitative polymerase chain reaction;

SEQ ID NO: 9 represents the sequence of a TaqMan probe used in the detection of a human Nox1 gene by real-time quantitative polymerase chain reaction;

SEQ ID NO: 10 represents the nucleotide sequence of siRNA for a human Nox1 gene of the present invention;

SEQ ID NO: 11 represents the nucleotide sequence of siRNA for a human Nox1 gene of the present invention;

SEQ ID NO: 12 represents the nucleotide sequence of siRNA for a rat Nox1 gene of the present invention;

SEQ ID NO: 13 represents the nucleotide sequence of siRNA for a rat Nox1 gene of the present invention;

SEQ ID NO: 14 represents the nucleotide sequence of siRNA for a rat Nox1 gene of the present invention;

SEQ ID NO: 15 represents the nucleotide sequence of a forward primer used in Example 1 for detecting the presence or absence of Nox1 expression by real-time quantitative polymerase chain reaction;

SEQ ID NO: 16 represents the nucleotide sequence of a reverse primer used in Example 1 for detecting the presence or absence of Nox1 expression by real-time quantitative polymerase chain reaction;

SEQ ID NO: 17 represents the nucleotide sequence of a TaqMan MGB probe used in Example 1 for detecting the presence or absence of Nox1 expression by real-time quantitative polymerase chain reaction;

SEQ ID NO: 18 represents the nucleotide sequence of an oligonucleotide that constitutes an siRNA construction used in Example 4 for examining the involvement of a Nox1 gene in transformation with a mutant Ras gene;

SEQ ID NO: 19 represents the nucleotide sequence of an oligonucleotide that constitutes an siRNA construction used in Example 4 for examining the involvement of a Nox1 gene in transformation with a mutant Ras gene;

SEQ ID NO: 20 represents the nucleotide sequence of an oligonucleotide that constitutes an siRNA construction used in Example 4 for examining the involvement of a Nox1 gene in transformation with a mutant Ras gene;

SEQ ID NO: 21 represents the nucleotide sequence of an oligonucleotide that constitutes an siRNA construction used in Example 4 for examining the involvement of a Nox1 gene in transformation with a mutant Ras gene;

SEQ ID NO: 22 represents the nucleotide sequence of an oligonucleotide that constitutes an siRNA construction used in Example 4 for examining the involvement of a Nox1 gene in transformation with a mutant Ras gene;

SEQ ID NO: 23 represents the nucleotide sequence of an oligonucleotide that constitutes an siRNA construction used in Example 4 for examining the involvement of a Nox1 gene in transformation with a mutant Ras gene;

SEQ ID NO: 24 represents the nucleotide sequence of an M13 primer used in Example 4 for confirming the transfection of the siRNA constructions for a Nox1 gene;

SEQ ID NO: 25 represents the nucleotide sequence of a 3.0 Rev primer used in Example 4 for confirming the transfection of the siRNA constructions for a Nox1 gene;

SEQ ID NO: 26 represents the nucleotide sequence of a forward primer used in Example 6 for confirming a decrease in Nox1 expression; and

SEQ ID NO: 27 represents the nucleotide sequence of a reverse primer used in Example 6 for confirming a decrease in Nox1 expression.

### REFERENCES

1. Suh, Y-A, et al. Nature 401, 79-82 (1999)
2. Arnold, R. S. et al. Proc. Natl. Acad. Sci. USA. 98, 5550-5555 (2001)
3. Arbiser, J. L. et al. Proc. Natl. Acad. Sci. USA. 99, 715-720 (2001)
4. Lassegue B. et al. Circ. Res. 88, 888-894 (2001)
5. Lambeth, J. D., Dheng, G., Arnold, R. S. & Edens, W. A. TIBS. 25, 459-461 (2000)
6. Royer-Pokora, B. et al. Nature 322, 32-38 (1986)
7. Kikuchi, H., Hikage, M., Miyashita, H. & Fulumoto, M. Gene 269, 131-140 (2001)

1. A composition for producing an antibody, comprising: (1) a polypeptide comprising the amino acid sequence of SEQ ID NO: 2; (2) a polypeptide having an amino acid sequence mutated from the amino acid sequence of SEQ ID NO: 2 by the substitution, deletion, addition, and/or insertion of one or more amino acid residues and inducing the production of an antibody specific to the polypeptide comprising the amino acid sequence of SEQ ID NO: 2; or (3) a polypeptide fragment having a partial sequence of the polypeptide of (1) or (2) and inducing the production of an antibody specific to the polypeptide comprising the amino acid sequence of SEQ ID NO: 2.

2. The composition according to claim 1, wherein the antibody is an antibody for detecting a human cancer cell.

3. The composition according to claim 1, comprising a polypeptide comprising the amino acid sequence of SEQ ID NO: 2.

4. The composition according to claim 1, comprising a polypeptide having a partial sequence of the amino acid sequence of SEQ ID NO: 2 and inducing the production of an antibody specific to the polypeptide comprising the amino acid sequence of SEQ ID NO: 2.

5. The composition according to claim 1, comprising a polypeptide fragment having a partial sequence of the amino acid sequence of SEQ ID NO: 2 and inducing the production of an antibody specific to the polypeptide comprising the amino acid sequence of SEQ ID NO: 2.

6. A method for producing an antibody specific to a polypeptide comprising the amino acid sequence of SEQ ID NO: 2, comprising administering a composition according to claim 1 to a mammal.

7. An antibody specific to a polypeptide comprising the amino acid sequence of SEQ ID NO: 2.

8. The antibody according to claim 7, wherein the antibody is a human/mouse chimeric antibody, a humanized antibody, or a human antibody.

9. The antibody according to claim 7, wherein the antibody is a polyclonal antibody or a monoclonal antibody.

10. A diagnostic method for cancer, comprising bringing an antibody according to claim 7 into contact with a biological sample.

11. A diagnostic kit for cancer, comprising an antibody according to claim 7.

12. A pharmaceutical composition for cancer therapy, comprising an antibody according to claim 7.

13. The pharmaceutical composition for cancer therapy according to claim 12, wherein the antibody is a human/mouse chimeric antibody, a humanized antibody, or a human antibody.

14. The pharmaceutical composition for cancer therapy according to claim 12, wherein the antibody is a polyclonal antibody or a monoclonal antibody.

15. The pharmaceutical composition for cancer therapy according to claim 12, further comprising an appropriate carrier.

16. A diagnostic method for cancer, characterized by detecting a polynucleotide comprising the nucleotide sequence of SEQ ID NO: 1 or a fragment thereof.

17. The diagnostic method for cancer according to claim 16, wherein the polynucleotide or the fragment thereof is detected by polymerase chain reaction (PCR) or real-time quantitative polymerase chain reaction.

18. The diagnostic method for cancer according to claim 17, wherein the detection is performed by PCR using a sense strand fragment corresponding to the nucleotide sequence of SEQ ID NO: 1 as a forward primer and an antisense strand fragment corresponding to the nucleotide sequence of SEQ ID NO: 1 as a reverse primer.

19. The diagnostic method for cancer according to claim 18, wherein the forward primer has 14 to 60 bases in length and the reverse primer has 14 to 60 bases in length.

20. The diagnostic method for cancer according to claim 18, wherein the diagnostic method uses the following forward and reverse primers:
(SEQ ID NO: 5)
the forward primer of 5′-GGAGCAGGAATTGGGGTCAC-3′;
and
(SEQ ID NO: 6)
the reverse primer of 5′-TTGCTGTCCCATCCGGTGAG-3′.

21. The diagnostic method for cancer according to claim 17, wherein the detection is performed by real-time quantitative polymerase chain reaction using a sense strand fragment corresponding to the nucleotide sequence of SEQ ID NO: 1 as a forward primer and an antisense strand fragment corresponding to the nucleotide sequence of SEQ ID NO: 1 as a reverse primer.

22. The diagnostic method for cancer according to claim 21, wherein the diagnostic method uses the following forward and reverse primers and TaqMan probe:
the forward primer of
(SEQ ID NO: 7)
5′-CCACTGTAGGCGCCCTAAGTT-3′;
the reverse primer of
(SEQ ID NO: 8)
5′-AAGAATGACCGGTGCAAGGA-3′;
and
the TaqMan probe of
(SEQ ID NO: 9)
5′-AAGGGCATCCCCCTGAGTCTTGGAA-3′.

23. siRNA corresponding to a polynucleotide comprising the nucleotide sequence of SEQ ID NO: 1 or a fragment thereof.

24. The siRNA according to claim 23, corresponding to a polynucleotide comprising a nucleotide sequence at positions from 71 to 1615 of SEQ ID NO: 1 or a fragment thereof.

25. The siRNA according to claim 23, wherein the siRNA has a nucleotide sequence from 8 to 30 bp in length.

26. The siRNA according to claim 23, wherein the siRNA consists of a nucleotide sequence selected from the group consisting of the following nucleotide sequences of SEQ ID NOs:10 to 14:
(SEQ ID NO: 10)
5′-GCGTGGCTTCAGCATGGAATTCAAGAGATTCCATGCTGAAGCCACGC
TTTTTTGGAAA-3′;
(SEQ ID NO: 11)
5′-GGGCTTTCGAACAACAATATTCAAGAGATATTGTTGTTCGAAAAGCC
CTTTTTTGGAAA-3′;
(SEQ ID NO: 12)
5′-GTTATGAGAAGTCTGACAAGTTCAAGAGACTTGTCAGACTTCTCATA
ATTTTTTGGAAA-3′;
(SEQ ID NO: 13)
5′-GATTCTTGGCTAAATCCCATTCAAGAGATGGGATTTAGCCAAGAATC
TTTTTTGGAAA-3′;
and
(SEQ ID NO: 14)
5′-GGACATTTGAACAACAGCATTCAAGAGATGCTGTTGTTCAAATGTCC
TTTTTTGGAAA-3′.

27. A pharmaceutical composition for cancer therapy, comprising: siRNA corresponding to a polynucleotide comprising the nucleotide sequence of SEQ ID NO: 1 or a fragment thereof; and siRNA corresponding to a polynucleotide comprising a nucleotide sequence at positions from 71 to 1615 of SEQ ID NO: 1 or a fragment thereof.

28. A pharmaceutical composition for cancer therapy, comprising a cell transformed with siRNA corresponding to a polynucleotide comprising the nucleotide sequence of SEQ ID NO: 1 or a fragment thereof; and siRNA corresponding to a polynucleotide comprising a nucleotide sequence at positions from 71 to 1615 of SEQ ID NO: 1 or a fragment thereof.

29. The pharmaceutical composition for cancer therapy according to claim 28, wherein the cell to be transformed is a cell taken out of a patient to be treated.

30. A method for producing a cell for cancer therapy, comprising preparing a human cell and transforming the cell with siRNA corresponding to a polynucleotide comprising the nucleotide sequence of SEQ ID NO: 1 or a fragment thereof; and siRNA corresponding to a polynucleotide comprising a nucleotide sequence at positions from 71 to 1615 of SEQ ID NO: 1 or a fragment thereof.

31. The method for producing a cell for cancer therapy according to claim 30, wherein the human cell is a cell taken out of a patient to be treated.

32. A screening method for a cancer cell growth inhibitor targeted for a Nox1 gene, comprising: transfecting a cell having a mutant Ras gene with a Nox1 gene; bringing the transformed cell into contact with a substance to be screened; and detecting the expression of the Nox1 gene and the inactivation of Nox1 activity.

33. The screening method according to claim 32, comprising culturing the transformed cell together with the substance to be screened.

34. The screening method according to claim 32, wherein the expression of the Nox1 gene is detected by detecting mRNA by real-time quantitative polymerase chain reaction or detecting a polypeptide or a peptide fragment thereof coded by the Nox1 gene with an antibody.

35. The screening method according to claim 32, wherein the expression of the Nox1 gene is detected by observing morphological changes in the transformed cell.

36. The screening method according to claim 32, wherein the cell having a mutant Ras gene is an H-Ras-NIH3T3 cell or a K-Ras-NRK cell.

37. The screening method according to claim 32, wherein the transfection of the Nox1 gene is performed using pEGFP-C1 (K-Ras-NRK/GFP) or pEGFP-C1-Nox1 (K-Ras-NRK/GFP-Nox1).

38. A cell having a mutant Ras gene, which is transfected with a Nox1 gene.
